(19) United States
(12) Patent Application Publication
(10) Pub. No.: US 2008/0021434 A1
(43) Pub. Date: Jan. 24, 2008

(54) Injection Port and Method of Making the Same

(75) Inventors: William A. Conard, Pennsburg, PA (US); Robert Buck, Watsontown, PA (US); Neil Strausbaugh, Williamsport, PA (US); Robert J. King, Malvern, PA (US); Diane M. Crammer, Phoenixville, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: WEST PHARMACEUTICAL SERVICES, INC., Lionville, PA (US)

(21) Appl. No.: 11/829,657

(22) Filed: Jul. 27, 2007

Related U.S. Application Data
(62) Division of application No. 11/137,188, filed on May 25, 2005.
(60) Provisional application No. 60/575,020, filed on May 27, 2004.

(51) Int. Cl. A61B 19/00

(52) U.S. Cl. 604/408

(57) ABSTRACT

An injection port for an intravenous bag including a generally hollow tube that is mountable to the intravenous bag. The hollow tube has a first end. A polymeric plug is mounted in the first end. The polymeric plug is integrally molded into the tube proximate the first end. A method for constructing the injection port including injecting a first molding material into a mold cavity, allowing the first molding material to at least partially cure and harden, moving a movable mold part to expose a first cavity defined by inner surfaces of the at least partially cured and hardened molding material, injecting a second mold material into the first cavity, allowing the second molding material to at least partially cure and harden such that the second molding material bonds with the inner surfaces and removing the injection port from the mold.

### CROSS-REFERENCE TO RELATED APPLICATIONS

This application is a divisional of co-pending U.S. application Ser. No. 11/137,188 filed May 25, 2005. This application claims the benefit of U.S. Provisional Application No. 60/575,020 filed May 27, 2004.

### BACKGROUND OF THE INVENTION

Intravenous (“IV”) bags typically include an injection port mounted to one of the walls of the bag that is used as a point of entry to inject medication or other liquids into the IV bag. The injection port is typically constructed of an injection molded, hollow polyvinyl chloride (“PVC”) tube with an elastomeric plug mounted to one end of the hollow PVC tube. The plug is mounted at an end of the hollow PVC tube by a shrink band that engages the plug and the hollow tube to hold the plug in position relative to the tube. The PVC tube is mounted to the IV bag such that the plug is facing out of the IV bag.

The typical injection port is constructed by molding the PVC tube, separately molding the plug and placing the plug onto a first end of the hollow PVC tube. The shrink band is then positioned around the sides of the plug and tube at their intersection and the shrink band is heated, which causes the shrink band to contract and secure the plug in the tube. The assembled injection port is inserted into a wall of the IV bag and is adhesively bonded to the wall.

The injection port is integral with the IV bag and seals or prevents leaks in the IV bag before, during and after a medication is injected into the bag using a syringe. The needle of the syringe is driven through the plug, which creates a seal with the needle to seal the IV bag, and medication is introduced into the IV bag through the needle. The needle may be inserted into and removed from the injection port multiple times without creating a permanent hole in the IV bag that would permit the contents of the IV bag to spill, because the plug creates a seal between itself and the needle and self-seals after the needle is withdrawn. The medication that is introduced into the IV bag is dispensed to a patient through an outlet port. The injection port may be utilized multiple times to introduce additional medication or other liquids into the IV bag without a leak forming in the injection port or the IV bag. However, the shrink wrap is prone to damage because it is exposed on an external surface of the injection port and may potentially permit the plug to release from the tube. In addition, because the plug is inserted into the tube following molding and curing, leaks may potentially form between the external surface of the plug and a mating surface of the tube.

The typical injection port is constructed using the above-described, three-step process of forming the hollow PVC tube, placing the plug onto the end of the tube and applying and heating the shrink band to the tube to secure the plug in the tube. A preferred injection port would reduce the labor intensive assembly process and reduce the steps required to construct the injection port while maintaining the sealing and self-sealing characteristics of the injection port.

### BRIEF SUMMARY OF THE INVENTION

Briefly stated, a preferred embodiment of the present invention comprises an injection port for an intravenous bag. The injection port includes a generally hollow tube that is mountable to the intravenous bag, wherein the hollow tube has a first end. A polymeric plug is mounted in the first end and is integrally molded into the tube.

In another aspect, a preferred embodiment of the present invention is directed to a method for constructing an injection port using a mold having a mold cavity and a movable mold part. The method includes the steps of injecting a first molding material into the mold cavity, allowing the molding material to at least partially cure and harden, moving the movable mold part from the mold cavity to expose a first cavity defined by inner surfaces of the at least partially cured and hardened molding material, injecting a second molding material into the first cavity, allowing the second molding material to at least partially cure and harden such that the second molding material bonds with the inner surfaces to form the injection port and removing the injection port from the mold.

In yet another aspect, a preferred embodiment of the present invention is directed to a method for constructing an injection port using a first mold having a first mold cavity and a second mold having a second mold cavity. The method includes the steps of injecting a first molding material into the first mold cavity, allowing the first molding material to at least partially cure and harden and removing the at least partially cured and hardened first mold material from the first mold such that the at least partially cured and hardened first mold material forms a hollow tube including a first cavity and a first end. The method also preferably includes the steps of injecting a second mold material into the second mold cavity, allowing the second molding material to at least partially cure and harden, removing the at least partially cured and hardened second mold material from the second mold, inserting the at least partially cured and hardened second mold material into the first cavity and mounting a cap onto the first end to secure the at least partially cured and hardened second mold material in the first cavity.

In a further aspect, a preferred embodiment of the present invention is directed to an intravenous bag for containing a fluid and permitting piercing of the bag with a needle to introduce additional fluid into the bag or to draw fluid out of the bag. The intravenous bag includes at least one wall constructed of a partially flexible material. The at least one wall defines a sealed cavity for containing the fluid. A hole is formed in the at least one wall and an injection port is mounted in the hole such that the fluid does not leak from the cavity through the hole. The injection port includes a generally hollow tube and a polymeric plug bonded to an inner surface of the tube.

### BRIEF DESCRIPTION OF THE SEVERAL VIEWS OF THE DRAWINGS

The foregoing summary, as well as the following detailed description of preferred embodiments of the invention will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 is a front elevational view of an injection port, in accordance with first and second preferred embodiments of the present application;

FIG. 1A is a cross-sectional view of the first preferred embodiment of the injection port shown in FIG. 1, taken along line 1A-1A of FIG. 1, wherein the injection port is mounted to an intravenous bag;

FIG. 2 is a top plan view of the injection port shown in FIG. 1;

FIG. 3 is a magnified, fragmentary view of a portion of the injection port shown in FIG. 1A, taken from within the dashed circle of FIG. 1A;

FIG. 4 is a cross-sectional view of a second preferred embodiment of the injection port shown in FIG. 1, taken along line 1A-1A of FIG. 1, wherein the injection port is mounted to an intravenous bag; and

FIG. 5 is a cross-sectional view of a tube of the injection port shown in FIG. 4.

### DETAILED DESCRIPTION OF THE INVENTION

Certain terminology is used in the following description for convenience only and is not limiting. The words “right”, “left”, “lower” and “upper” designate directions in the drawings to which reference is made. The words “inwardly” and “outwardly” refer to directions toward and away from, respectively, the geometric center of the preferred embodiments of the injection port and designated parts thereof. The terminology includes the above-listed words, derivatives thereof and words of similar import. Additionally, the word “a” as used in the specification means “at least one”.

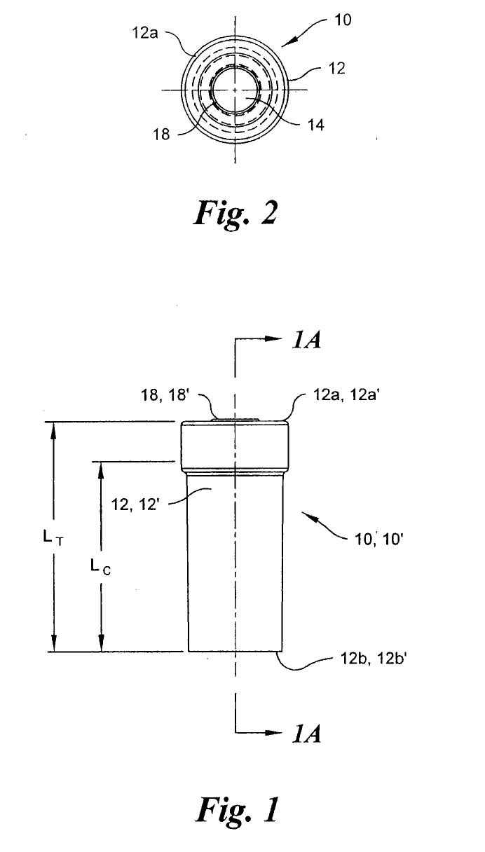

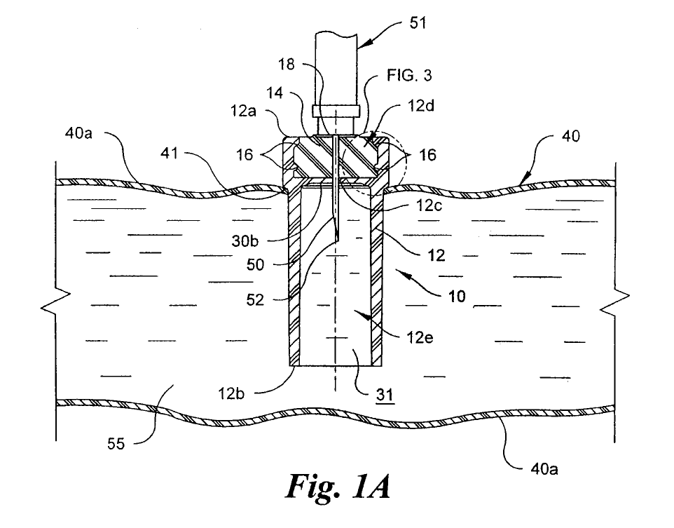

Referring to FIGS. 1-3, a first preferred embodiment an injection port, generally designated 10, includes a generally hollow tube 12 and a polymeric plug 14. The tube 12 is preferably constructed of an injection molded polyvinyl chloride (“PVC”) material and the plug 14 is preferably constructed of a thermoplastic elastomer (“TPE”) material. The PVC material of the tube 12 is preferred for its formability, flexibility, ability to be mounted to an IV bag 40 in a liquid-tight manner and additional properties that are obvious to one having ordinary skill in the art. The TPE material is preferred for the plug 14 for its self-sealing properties, formability and additional properties that are obvious to one having ordinary skill in the art and is described in greater detail below. One having ordinary skill in the art will realize that the tube 12 is not limited to flexible, injection molded PVC materials and the plug 14 is not limited to TPE materials. The tube 12 and plug 14 may be constructed of nearly any material that is able to take on the general shape, perform the functions and withstand the operating conditions of the tube 12 and plug 14, respectively.

In the first preferred embodiment, the tube 12 has a first end 12a, a second end 12b and a diaphragm 12c that spans the hollow tube 12 at a predetermined location between the first and second ends 12a, 12b. The diaphragm 12c preferable includes a first side 30a that faces the first end 12a and a second side 30b that faces the second end 12b. A first cavity 12d is preferably defined by the diaphragm 12c and the first end 12a and is more specifically, preferably defined by the first side 30a, the first end 12a and inner surfaces 31 of the tube 12. A second cavity 12e is preferably defined by the diaphragm 12c and the second end 12b and is more specifically, preferably defined by the second side 30b, the second end 12b and the inner surfaces 31 of the tube 12. The first cavity 12d is preferably filled by the plug 14 and the second cavity 12e is preferably empty in an assembled configuration of the injection port 10.

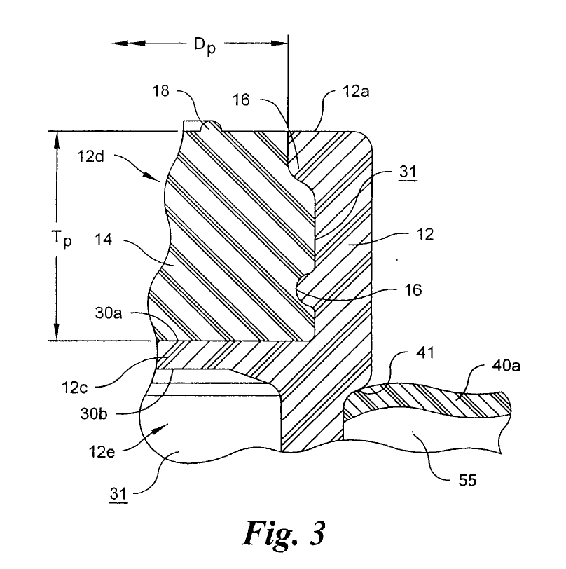

Referring to FIGS. 1A and 3, the tube 12 of the first preferred embodiment includes ribs 16 that extend around the inner surface 31 into the first cavity 12d. The ribs 16 provide a bonding surface for the plug 14 when it is injected into the first cavity 12d. In the first preferred embodiment, the plug 14 is preferably bonded to the inner surface 31b, ribs 16 and first side 30a of the diaphragm 12c, as will be described in greater detail below. The ribs 16 also provide a structural impediment that secures the plug 14 in the first cavity 12d. Two ribs 16 preferably extend around the inner surface 31 of the first cavity 12d and have an arcuate-shape. The arcuate-shape of the ribs 16 provides a surface for bonding with the plug 14 and is advantageous for manufacturing the tube 12. The ribs 16 are not limited to the arcuate-shape, the above-identified number or to being included in the first cavity 12d. For example, the first cavity 12d may include no ribs or may include several cylindrical-shaped ribs that extend into the first cavity 12d, generally perpendicularly to the inner surface 31.

Referring to FIGS. 1-3, the plug 14 of the first preferred embodiment has a plug diameter D_{P} of approximately two tenths of an inch (0.2″) and a plug thickness T_{P} of approximately twelve tenths of an inch (0.12″). The plug 14, having these preferred dimensions, is typically able to withstand at least one hundred (100) penetrations without a significant decrease in performance and self-sealing properties. However, one having ordinary skill in the art will realize that the plug 14 is not limited to the above-listed dimensions and may have nearly any shape and/or size that is required for a specific injection port application. For example, the plug 14 may have a generally cubic-shape to fit into a generally cubic-shaped first cavity 12d and may be relatively larger or smaller than the preferred plug 14.

The plug 14 of the first preferred embodiment includes a target ring 18 that has a ring-shape and extends from a surface of the plug 14 opposite the diaphragm 12c in the assembled configuration. The target ring 18 is preferably integrally molded with the plug 14 and provides a target within which a user preferably punctures the plug 14. That is, the plug 14 is preferably punctured by a needle 50 within the target ring 18 such that the needle 50 is driven through the plug 14, through the diaphragm 12c and into the second cavity 12e during use. It is preferable that the needle 50 extend through the plug 14, diaphragm 12c and into the second cavity 12e so that the liquid from a syringe 51 is injected into the IV bag 40. Conversely, it is undesirable for the needle 50 to extend through the plug 14 and then through the tube 12 somewhere other than the diaphragm 12c because the IV bag 40 or tube 12 may be compromised and the contents of the IV bag 40 may leak or become contaminated, as will be understood by one having ordinary skill in the art.

In addition, the second cavity 12e preferably has a relatively long cavity length Lc when compared to the plug thickness T_{P}. The cavity length L_{C} is preferably relatively long such that when the needle 50 is inserted through the plug 14 and diaphragm 12c, the tip 52 does not puncture an opposite wall of the IV bag 50 that the injection port 10 is mounted in, as will also be understood by one having ordinary skill in the art. In the preferred embodiments, the cavity length L_{C} is approximately forty-five tenths of an inch (0.45″). However, the cavity length Lc is not limited to being longer than the plug thickness T_{P} or to the specifically identified length and may be shorter or longer depending upon the application.

The assembled injection port 10 of the first preferred embodiment is preferably produced by a rotational injection molding process. Specifically, an injection mold (not shown) having a mold cavity in the shape of the tube 12 is positioned in a machine and a first mold material is injected into the mold to form the hollow tube 12. The first mold material is preferably comprised of liquefied PVC. The liquefied PVC is given time to cool and harden, the mold is moved or rotated to a second position and a movable mold part is moved from the mold cavity to expose the first cavity 12d of the tube 12. A second mold material preferably comprised of liquefied TPE is injected into the first cavity 12d, preferably to form the plug 14 in the first cavity 12d. The liquefied TPE is given time to cool, harden and bond to the inner surfaces 31 of the first cavity 12d and the ribs 16. The mold is disassembled and the co-molded, one-piece injection port 10 is removed from the mold. One having ordinary skill in the art will realize that the injection port 10 is not limited to the above-described manufacturing method or steps and may be produced using nearly any method or process that is able to produce the injection port 10 including the hollow tube 12 and plug 14. For example, the injection port 10 may be manufactured using a rotary compression or transfer molding operation or may be formed by machining the tube 12 from a metallic material and forming the plug 14 by inserting or molding nearly any self-sealing material in the first cavity 12d.

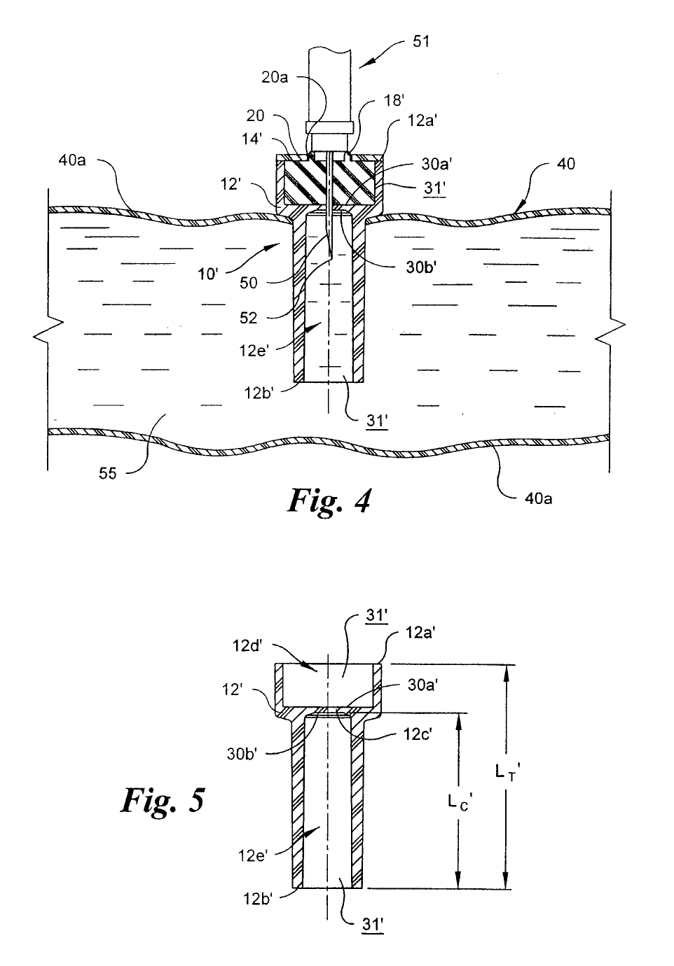

Referring to FIGS. 1, 4 and 5, a second preferred embodiment of the injection port, generally designated 10′, has a similar construction as the injection port 10 of the first preferred embodiment. Like reference numerals are utilized in FIGS. 1, 4 and 5 to indicate like elements or components of the injection port 10′ of the second preferred embodiment when compared to elements or components of the injection port 10 of the first preferred embodiment with a (′) symbol utilized to identify like elements or components of the second preferred embodiment.

The injection port 10′ of the second preferred embodiment includes a disc-shaped cap 20 with a central hole 20a therein. The cap 20 is bonded to the first end 12a′ of the tube 12′ to further secure the plug 14′ in the first cavity 12d′ in the assembled condition. The hole 20a accommodates insertion of the needle 50 into the plug 14′ during injection. The hollow tube 12′ of the second preferred embodiment also includes the first cavity 12d′, however, the first cavity 12d′ has a generally cylindrical-shape with generally smooth inner surfaces 31′ (i.e. no ribs 16).

The injection port 10′ is constructed by injecting a first molding material, which is preferably comprised of liquefied PVC, into a first mold, allowing time for the first molding material to cure and harden and removing the at least partially cured and hardened first mold material from the first mold. The at least partially cured and hardened first mold material preferably forms the hollow tube 12′. A second molding material, which is preferably comprised of liquefied TPE is injected into a second mold, the second mold materials is allowed time to at least partially cure and harden and the second mold material is removed from the second mold. The at least partially cured and hardened second mold material preferably forms the plug 14′. The plug 14′ is preferably inserted into the first cavity and the cap 20 is mounted onto the first end 12a′ of the tube 12′ to secure the plug 14′ in the first cavity 12d′. The cap 20 may be adhesively bonded, clamped, ultrasonically welded or otherwise secured to the first end 12a′ to secure the cap 20 to the first end 12a′ and to secure the plug 14 within the first cavity 12d′.

In the preferred embodiments, the injection port 10, 10′ is mounted to a wall 40a of the IV bag 40 such that the bag 40 is sealed and is able to contain a fluid 55. The wall 40a is preferably constructed of a partially flexible material and defines a sealed cavity for containing the fluid 55. The wall 40a includes a hole 41 therein and the injection port 10, 10′ is mounted in the hole 41 such that the fluid 55 generally does not leak from the cavity through the hole 41. The injection port 10, 10′ preferably provides a port through which the needle 50 of the syringe 51 may be inserted to inject fluid into or withdraw fluid from the IV bag 40. In the preferred embodiment, the injection port 10, 10′ and specifically, the hollow tube 12, 12′ has a tube length L_{T} that is greater than a length of the needle 50. The configuration generally prevents the needle tip 52 from coming into contact with the wall 40a and potentially puncturing the wall 40a and the IV bag 40.

To assemble the injection port 10, 10′ with the IV bag 40, the second end 12b, 12b′ is inserted into the hole 41 in the wall 40. The wall 40a is adhesively bonded to the tube 12, 12′ such that a liquid seal is created between the tube 12, 12′ and the wall 40a. The bond between the tube 12, 12′ and wall 40a is not limited to adhesive bonding and may be comprised of ultrasonic welding, heat sealing or other like bonding methods, as long as a liquid-tight seal is created between the tube 12, 12′ and the wall 40a, such that the fluid 55 of the IV bag 40 does not leak.

In operation, the needle 50 of the syringe 51 is urged through the plug 14, 14′, preferably within the diameter of the target ring 18, 18′, through the diaphragm 12c, 12c′ and into the second cavity 12e, 12e′, which is exposed to an inside of the IV bag 40 and the fluid 55. The second end 12b, 12b′ spaces the diaphragm 12c, 12c′ and the needle 50 from the opposite wall 40a of the IV bag 40 such that the wall 40a is not punctured by the needle 50 and the medication or liquid dispensed from the needle 50 is introduced into the IV bag 40. The plug 14, 14′ creates a seal between itself and the needle 50 such that liquid or medication 55 from inside the IV bag 40 is unable to leak from the bag 40 between the needle 50 and plug 14, 14′. The needle 50 is removed from the injection port 10, 10′, leaving a hole in the diaphragm 12c, 12c′. The ribs 16 and the cap 20 provide a retaining force to secure the plug 14, 14′ within the first cavity 12d, 12d′ while the needle 50 is removed from the plug 14, 14′. The TPE material of the plug 14, 14′ self-seals such that liquid or medication from inside the IV bag 40 does not leak through the hole in the diaphragm 12c, 12c′ when the needle 50 is removed from the injection port 10, 10′.

In the preferred embodiments, the TPE material utilized for the plug 14 is preferably comprised of a styrenic block copolymer having a Shore A hardness of about twenty-five (25) to about ninety (90), more preferably about thirty (30) to about forty-five (45), and a compression set less than about fifty-five percent (55%). The styrenic block copolymer has a preferred ratio of styrene segments to midblock (rubber) segments of about twenty-eight to thirty-seven percent (28-37%) styrene to about sixty-three to seventy-two percent (63-72%) midblock. More preferably, the block copolymer comprises about thirty percent (30%) styrene to about seventy percent (70%) midblock, and most preferably comprises about thirty-three percent (33%) styrenic segments and about sixty-seven percent (67%) midblock segments.

The presently preferred styrenic block copolymer is one comprising ethylene and butylene midblock segments (SEBS) because these polymers are autoclavable and, due to their saturated midblocks, are able to withstand environmental harassment. For example, SEBS polymers do not crack under UV light. Other appropriate midblock components include, but are not limited to, isoprene, isobutylene, butadiene, and propylene. Styrenic block copolymers containing these segments are well known in the art as SEEPS, SIBS, SBS, SIS, and SEPS, for example and are commercially available under the tradenames Kraton® and Septon™ (manufactured by Kuraray). Styrenic block copolymers containing saturated midblocks are preferred because they are less likely to be attacked by environmental radiation. It is also within the scope of the invention to include more than one styrenic block copolymer in the TPE.

The styrenic block copolymer may be blended with other ingredients to provide desired properties to the plug. Appropriate ingredients include, but not are limited to, plasticizers, thermoplastics, antioxidants, fillers, coloring agents, processing aids, and other conventional additives known in the art.

For example, an exemplary plug material for use in the invention has the composition shown in the Table below. Concentrations are expressed as phr (parts per hundred rubber). As previously explained, the presently preferred styrenic block copolymer is an SEBS polymer. Other preferred components include a hydrocarbon oil, a phenolic antioxidant and a polypropylene or polyethylene having a melt flow between about five (5) and about fifty (50). However, specific components may be determined by routine experimentation depending on the particular styrenic block copolymer which is included in the TPE.

| | possible | preferred |
|---|---|---|
| Component | concentration (phr) | concentration (phr) |
| styrenic block | 5-100 | 80-100 |
| copolymer | | |
| oil (plasticizer) | 5-160 | 100-150 |
| polypropylene or | 5-40 | 15-30 |
| polyethylene | | |
| antioxidants | 1-4 | 0.2 |
| pigment (coloring | 1-4 | 0.2 |
| agent) | | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An intravenous bag for containing a fluid and permitting piercing of the bag with a needle to at least one of introduce additional fluid into the bag and to draw fluid out of the bag, the intravenous bag comprising:
at least one wall constructed of a partially flexible material, the at least one wall defining a sealed cavity for containing the fluid;
a hole in the at least one wall; and
an injection port mounted in the hole such that the fluid does not leak from the cavity through the hole, the injection port including a generally hollow tube and a polymeric plug bonded to an inner surface of the tube.

2. The intravenous bag of claim 1 wherein the polymeric plug comprises a thermoplastic elastomer.

3. The intravenous bag of claim 1 further comprising:
a diaphragm positioned within the hollow tube between first and second ends, a first cavity defined by the inner surface, first end and diaphragm and a second cavity defined by the inner surface, second end and diaphragm, the polymeric plug located within the first cavity.

4. The intravenous bag of claim 1 wherein the hollow tube has a tube length that is greater than a length of the needle.
